SEQUENCE LISTING (23 sequences)

SEQ ID NO: 1 - RNA, 22 nt, Artificial sequence
  micro RNA nucleic acid sequence
uuaucagaaucuccagggguac

SEQ ID NO: 2 - RNA, 21 nt, Artificial sequence
  micro RNA nucleic acid sequence
uggguuuacguugggagaacu

SEQ ID NO: 3 - RNA, 21 nt, Artificial sequence
  micro RNA nucleic acid sequence
ucuaguaagaguggcagucga

SEQ ID NO: 4 - RNA, 21 nt, Artificial sequence
  micro RNA nucleic acid sequence
ugguagacuauggaacguagg

SEQ ID NO: 5 - RNA, 22 nt, Artificial sequence
  micro RNA nucleic acid sequence
uggaauguaaagaaguauguau

SEQ ID NO: 6 - RNA, 22 nt, Artificial sequence
  micro RNA nucleic acid sequence
uucaaguaauccaggauaggcu

SEQ ID NO: 7 - RNA, 24 nt, Artificial sequence
  micro RNA nucleic acid sequence
ucccugagacccuuuaaccuguga

SEQ ID NO: 8 - RNA, 22 nt, Artificial sequence
  micro RNA nucleic acid sequence
ucccugagacccuaacuuguga

SEQ ID NO: 9 - RNA, 21 nt, Artificial sequence
  micro RNA nucleic acid sequence
cauaaaguagaaagcacuacu

SEQ ID NO: 10 - RNA, 22 nt, Artificial sequence
  micro RNA nucleic acid sequence
uuauaaagcaaugagacugauu

SEQ ID NO: 11 - RNA, 20 nt, Artificial sequence
  micro RNA nucleic acid sequence
ccucugggcccuuccuccag

SEQ ID NO: 12 - RNA, 21 nt, Artificial sequence
  micro RNA nucleic acid sequence
ucacagugaaccggucucuuu

SEQ ID NO: 13 - RNA, 23 nt, Artificial sequence
  micro RNA nucleic acid sequence
ugaggggcagagagcgagacuuu

SEQ ID NO: 14 - RNA, 23 nt, Artificial sequence
  micro RNA nucleic acid sequence
agcagcauuguacagggcuauca

SEQ ID NO: 15 - RNA, 23 nt, Artificial sequence
  micro RNA nucleic acid sequence
aucacauugccagggauuaccac

SEQ ID NO: 16 - RNA, 20 nt, Artificial sequence
  micro RNA nucleic acid sequence
aaaagcuggguugagagggu

SEQ ID NO: 17 - RNA, 22 nt, Artificial sequence
  micro RNA nucleic acid sequence
ccaccuccccugcaaacgucca

SEQ ID NO: 18 - RNA, 22 nt, Artificial sequence
  micro RNA nucleic acid sequence
cuauacgaccugcugccuuucu

SEQ ID NO: 19 - RNA, 22 nt, Artificial sequence
  micro RNA nucleic acid sequence
acuggacuuggagucagaaggc

SEQ ID NO: 20 - RNA, 23 nt, Artificial sequence
  micro RNA nucleic acid sequence
agcagcauuguacagggcuauga

SEQ ID NO: 21 - RNA, 22 nt, Artificial sequence
  micro RNA nucleic acid sequence
ucagugcacuacagaacuuugu

SEQ ID NO: 22 - RNA, 22 nt, Artificial sequence
  micro RNA nucleic acid sequence
gaauguugcucggugaaccccu

SEQ ID NO: 23 - RNA, 22 nt, Artificial sequence
  micro RNA nucleic acid sequence
ucagugcaucacagaacuuugu

(19) United States
(12) Patent Application Publication
(10) Pub. No.: US 2022/0364175 A1
(43) Pub. Date: Nov. 17, 2022

(54) DIAGNOSIS OF FRONTOTEMPORAL DEMENTIA

(71) Applicants: Yeda Research and Development Co. Ltd., Rehovot (IL); UCL Business Ltd, London (GB)

(72) Inventors: Eran HORNSTEIN, Rehovot (IL); Iddo MAGEN, Rehovot (IL); Nancy-Sarah YACOVZADA, Rehovot (IL); Jonathan ROHRER, London (GB); Pietro FRATTA, London (GB)

(73) Assignees: Yeda Research and Development Co. Ltd., Rehovot (IL); UCL Business Ltd, London (GB)

(21) Appl. No.: 17/846,157

(22) Filed: Jun. 22, 2022

Related U.S. Application Data
(63) Continuation of application No. PCT/IL2020/051323, filed on Dec. 22, 2020.
(60) Provisional application No. 62/952,354, filed on Dec. 22, 2019.

(51) Int. Cl. C12Q 1/6883; G01N 33/68

(52) U.S. Cl. CPC C12Q 1/6883; G01N 33/68; C12Q 2600/178; C12Q 2600/158

(57) ABSTRACT

A method of diagnosing Frontotemporal dementia (FTD) in a subject in need thereof is provided. The method comprising detecting a level of at least one micro RNA (miR) selected from the group consisting of hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p and hsa-miR-340-5p in a biological sample of the subject, wherein when said level of said at least one micro RNA (miR) is higher than that in a control sample, it is indicative of FTD.

### RELATED APPLICATIONS

This application is a Continuation of PCT Patent Application No. PCT/IL2020/051323 having International filing date of Dec. 22, 2020, which claims the benefit of priority under 35 USC § 119(e) of U.S. Provisional Patent Application No. 62/952,354 filed on Dec. 22, 2019. The contents of the above applications are all incorporated by reference as if fully set forth herein in their entirety.

### SEQUENCE LISTING STATEMENT

The ASCII file, entitled 92503SequenceListing.txt, created on Jun. 22, 2022, comprising 5,068 bytes, submitted concurrently with the filing of this application is incorporated herein by reference.

### FIELD AND BACKGROUND OF THE INVENTION

The present invention, in some embodiments thereof, relates to the diagnosis of Frontotemporal dementia (FTD).

FTD is a clinically and neuroanatomically heterogeneous neurodegenerative disorder characterized by frontal and temporal lobe atrophy. It typically manifests between the ages of 50 and 70 with behavioral or language problems, and below the age of 65 is the second most common form of dementia, after Alzheimer's disease (1).

Due to heterogeneity in clinical presentation, FTD can be difficult to diagnose (2). Three main phenotypes are described: behavioral variant frontotemporal dementia (bvFTD), characterized by changes in social behaviour and conduct, semantic dementia (SD), characterized by the loss of semantic knowledge, leading to impaired word comprehension, and progressive non-fluent aphasia (PNFA), characterized by progressive difficulties in speech production (2, 3).

FTD is also pathologically heterogeneous with inclusions seen containing hyperphosphorylated tau (4), TDP-43 (5), or fused in sarcoma (FUS) (6, 7). Mutations in the genes encoding for these proteins, as well as in other genes such as progranulin (GRN), chromosome 9 open reading frame 72 (C9ORF72), valosin-containing protein (VCP), TANK-binding kinase 1 (TBK1) and charged multivesicular body protein 2B (CHMP2B) are also associated with FTD (8-11).

FTD overlaps clinically, pathologically and genetically with several other degenerative disorders. In particular, there is often overlap with amyotrophic lateral sclerosis (ALS): one in 5 ALS patients meets the clinical criteria for a concomitant diagnosis of FTD, and one in eight FTD patients is also diagnosed with ALS. TDP-43 inclusions are observed in the brains of both people with FTD and ALS, and genetic evidence supports that these diseases reside along a continuum (5, 12-14).

Previous studies have aimed to develop cell-free biomarkers for FTD, including TDP-43 (15), tau (16), and neurofilament light chain (Nf) (17), but none of these have shown use for diagnosis. microRNAs (miRNAs), endogenous non-coding RNAs, can be quantified in biofluids (18), and have been shown previously to be dysregulated in amyotrophic lateral sclerosis (ALS) and in FTD (19). Furthermore, they may be biomarkers of disease progression in other brain diseases, including ALS (20). Previous studies have assessed the initial potential of microRNAs as diagnostic FTD biomarkers including miRNA analysis in plasma (21, 22), CSF and serum (23), and CSF exosomes (24) but no definitive diagnostic markers have so far been found.

### SUMMARY OF THE INVENTION

According to an aspect of some embodiments of the present invention there is provided a method of diagnosing Frontotemporal dementia (FTD) in a subject in need thereof, the method comprising detecting a level of at least one micro RNA (miR) selected from the group consisting of hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p and hsa-miR-340-5p in a biological sample of the subject, wherein when the level of the at least one micro RNA (miR) is higher than that in a control sample, it is indicative of FTD.

According to an aspect of some embodiments of the present invention there is provided a method of treating Frontotemporal dementia (FTD) in a subject in need thereof, the method comprising:

(a) diagnosing FTD according to claim 1;

(b) treating the subject with a drug which ameliorates symptoms associated with FTD.

According to an aspect of some embodiments of the present invention there is provided a method of treating Frontotemporal dementia (FTD) in a subject in need thereof, the method comprising: treating the subject with a drug which ameliorates symptoms associated with FTD, wherein the subject has been diagnosed with FTD according to the method of claim 1.

According to an aspect of some embodiments of the present invention there is provided a method for selecting subjects for enrollment in a clinical trial involving treatment of Frontotemporal dementia (FTD), the method comprising:

(a) determining diagnosis of a subject according to claim 1; and

(c) identifying the subject as being suitable for the clinical trial based on the criteria of the clinical trial.

According to some embodiments of the invention, the at least one miR comprises a range selected from the group consisting of 2-24, 2-20, 2-18, 2-16, 2-14, 2-12, 2-10, 2-8, 2-6 and 2-4.

According to some embodiments of the invention, at least one additional micro RNA (miR) is selected from the group consisting of hsa-miR-326, hsa-miR-128-3p, hsa-miR-423-5p, hsa-miR-107, hsa-miR-23b-3p, hsa-miR-320c, hsa-miR-1306-5p, hsa-let-7d-3p, hsa-miR-378a-3p, hsa-miR-103a-3p, hsa-miR-148a-3p, hsa-miR-409-3p and hsa-miR-148b-3p

According to some embodiments of the invention, the at least one micro RNA (miR) comprise hsa-miR-361-5p and hsa-miR-629-5p.

According to some embodiments of the invention, the at least one micro RNA (miR) comprises hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p, hsa-miR-340-5p, hsa-miR-326, hsa-miR-128-3p, hsa-miR-423-5p, hsa-miR-107, hsa-miR-23b-3p, hsa-miR-320c, hsa-miR-1306-5p, hsa-let-7d-3p, hsa-miR-378a-3p, hsa-miR-103a-3p, hsa-miR-148a-3p, hsa-miR-409-3p and hsa-miR-148b-3p,

According to some embodiments of the invention, the at least one micro RNA (miR) comprise, miR-1-3p, miR-361-5p, miR-629-5p, miR-423-5p,

According to some embodiments of the invention, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p and miR-23b-3p.

According to some embodiments of the invention, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p, miR-23b-3p, miR-326 and miR-103a-3p.

According to some embodiments of the invention, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p, miR-23b-3p, miR-326, miR-103a-3p, miR-142-5p, miR-125a-5p, miR-26a-5p, miR-148a-3p, miR-379-5p and miR-320c.

According to some embodiments of the invention, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p, miR-23b, miR-326, miR-103a-3p, miR-142-5p, miR-125a-5p, miR-26a-5p, miR-148a-3p, miR-379-5p, miR-320c, let-7d-3p, miR-340-5p and miR-1306-5p.

According to some embodiments of the invention, the at least one micro RNA (miR) is selected from the group consisting of hsa-miR-361-5p, hsa-miR-629-5p, hsa-miR-26a-5p, hsa-miR-23b-3p, hsa-miR-326, hsa-miR-103a-3p, hsa-miR-379-5p, hsa-miR-320c, hsa-miR-128-3p, hsa-miR-148a-3p, hsa-miR-628-3p, hsa-miR-423-5p and hsa-let-7d-3p.

According to some embodiments of the invention, the at least one micro RNA (miR) comprise hsa-miR-361-5p, hsa-miR-629-5p, hsa-miR-26a-5p, hsa-miR-23b-3p, hsa-miR-326, hsa-miR-103a-3p, hsa-miR-379-5p, hsa-miR-320c, hsa-miR-128-3p, hsa-miR-148a-3p, hsa-miR-628-3p, hsa-miR-423-5p and hsa-let-7d-3p.

According to some embodiments of the invention, the method further comprises retrieving the biological sample from the subject.

According to some embodiments of the invention, the biological sample is cell-free.

According to some embodiments of the invention, the biological sample is selected from the group consisting of a plasma, a serum and a cerebrospinal fluid sample.

According to some embodiments of the invention, the detecting is effected by real time PCR (RT-PCR).

According to some embodiments of the invention, the detecting is effected by next generation sequencing (NGS).

According to some embodiments of the invention, the subject is a human being.

According to some embodiments of the invention, the at least one micro RNA (miR) does not exceed 100 miRs.

According to some embodiments of the invention, the at least one micro RNA (miR) does not exceed 50 miRs.

According to some embodiments of the invention, the at least one micro RNA (miR) does not exceed 30 miRs.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G show diagnosis of FTD by a cell free miRNA signature. FIG. 1A—Predictor capacity to diagnose FTD based on circulating miRNA, assessed as 91%-95% (mean 93%) (0.91-0.95 (mean 0.93) by receiving operating characteristic (ROC) curves in the training set, that were split into three subsets (3-fold cross validation) of the area under the curve (AUC), noting a confidence interval [CI of 91%-95%]. FIG. 1B—Performance and generalizability proven on held-out data, that serves for verifications with 91% (0.91) success. Given a single probability threshold of 70%, the classifier is yielding an 0.85 True Positive Rate with 0.13 False Positive Rate. FIG. 1C—Histogram of binned predicted values and reliability diagram for 5 data bins. True fraction of positive cases plotted against predicted values. Most values predicted by the boosted trees are approaching near 0 or 1, while only few predictions lie in the central region (bottom row). There is a sharp drop in the number of cases predicted to have probability between 0.2 to 0.5. FIG. 1D—Precision-recall curve is demonstrating the trade-off between True Positive Rate and the Positive Predictive Value (PPV). An average precision of 0.89 in the held-out set is obtained. FIG. 1E—Confusion matrix in held-out set. FIG. 1F—Cross validated Recursive Feature Elimination (CV-RFE) reduced the number of miRNAs features from 134 to 23. Among the most predictive features are miR-107 (implicated in Alzheimer's disease pathogenesis) and miRNAs expressed in the brain, such as miR-125a-5p and miR-26a-5p. FIG. 1G—AUC ROC of a model trained with only a subset of top k features (1 to 23 most predictive miRNAs), show stability in performance with the selected final features.

FIGS. 2A-B break down the contribution of individual features revealing disease-specific miRNA predictors. FIG. 2A—SHapley Additive exPlanations (SHAP) analysis describes the impact of specific features on FTD disease predictor output. Average impact (mean absolute SHAP values) of miRNAs on output, according to importance. FIG. 2B—Illustration of the relationship between the value of miRNA features from low (blue) to high (red) and the impact on the prediction; each point is a SHAP value for a feature and a specific subject.

### DESCRIPTION OF SPECIFIC EMBODIMENTS OF THE INVENTION

The present invention, in some embodiments thereof, relates to diagnosis of Frontotemporal dementia (FTD).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Frontotemporal dementia (FTD) is a heterogeneous neurodegenerative disorder characterized by frontal and temporal lobe atrophy, typically manifesting with behavioural or language impairment. Because of its heterogeneity and lack of available diagnostic laboratory tests there can be a substantial delay in diagnosis.

Whilst conceiving embodiments of the invention and reducing it to practice, the present inventors uncovered selected microRNAs as biomarkers for FTD diagnosis.

As is described in the Examples section which follows, the present inventors enrolled a large cohort of patients diagnosed with FTD (n=168) with different clinical phenotypes and pathological forms, as well as healthy controls (n=125) and performed next generation small RNA sequencing on cell-free plasma in these individuals in an unbiased manner. Based on the miRNA profile, the present inventors developed novel prediction models by implementing machine learning algorithms that are able to correctly distinguish FTD from controls with accurate diagnosis >90%. The analysis revealed the diagnostic importance of circulating endogenous miRNAs in distinguishing FTD cases from controls. Thus, microRNAs of some embodiments of the invention are promising FTD biomarkers that might improve accurate identification of patients for clinical trials.

Thus, according to an aspect of the invention there is provided a method of diagnosing Frontotemporal dementia (FTD) in a subject in need thereof, the method comprising detecting a level of at least one micro RNA (miR) selected from the group consisting of hsa-miR-361-5p, hsa-miR-629-5p, hsa-miR-107, hsa-miR-378a-3p, hsa-miR-26a-5p, hsa-miR-1-3p, hsa-miR-23b-3p, hsa-miR-340-5p, hsa-miR-326 and hsa-miR-142-5p in a biological sample of the subject, wherein when said level of said at least one micro RNA (miR) is higher than that in a control sample, it is indicative of FTD.

As used herein “Frontotemporal dementia” or “FTD”, is a neurodegenerative disease characterized by severe frontotemporal lobar degeneration. FTD is distinguished from Alzheimer's disease and Lewy body dementia based on several factors one of which being that it does not manifest with amyloid plaques, neurofibrillary tangles, or Lewy bodies. Symptoms of FTD typically appear around 45 to 65 years of age. Symptoms typically progress at a rapid, steady rate. Some patients with FTD also exhibit motor neuron disease (MND) e.g. ALS (i.e. FTD-ALS).

Typically, symptoms are classified into three groups based on the functions of the frontal and temporal lobes: (1) Behavioral variant FTD (bvFTD) exhibits symptoms of lethargy and aspontaneity on the one hand, and disinhibition on the other. Apathetic patients may become socially withdrawn and stay in bed all day or no longer take care of themselves. Disinhibited patients can make inappropriate (sometimes sexual) comments or perform inappropriate acts (e.g. stealing or speeding). (2) Progressive nonfluent aphasia (PNFA), also referred to as nonfluent variant primary progressive aphasia (nfvPPA), presents with a breakdown in speech fluency due to articulation difficulty, phonological and/or syntactic errors but preservation of word comprehension. (3) Semantic dementia (SD), also referred to as semantic variant primary progressive aphasia (svPPA), can be found in some patients that remain fluent with normal phonology and syntax, but increasing difficulty with naming and word comprehension. It has been researched that some may even go through depression and lose their inhibitions and exhibit antisocial behavior.

As used herein “diagnosing” refers to determining presence or absence of a pathology (i.e., FTD), classifying a pathology or a symptom, determining a severity of the pathology, monitoring pathology progression, forecasting an outcome of a pathology and/or prospects of recovery and screening of a subject for a specific disease.

According to some embodiments of the invention, screening of the subject for a specific disease is followed by substantiation of the screen results using gold standard methods.

Some Gold standard methods for diagnosing FTD include:

FTD diagnosis may be affected using the criteria proposed by the international consortium in 2011. A summary of these criteria can be found in Bott et al., Neurodegener Dis Manag. (2014) 4(6): 439-454, incorporated herein by reference in its entirety.

In short, diagnosis of bvFTD typically requires a patient to have a progressive deterioration of behavior accompanied by three out of six core features (disinhibition, apathy, loss of sympathy/empathy, eating behavior changes, compulsive behaviors and an executive predominant pattern of dysfunction on cognitive testing). Additionally, functional decline and neuroimaging consistent with bvFTD are used for diagnosis. Neuroimaging findings include e.g. frontal, or anterior temporal atrophy, or both, on CT or MRI, or frontal hypoperfusion or hypometabolism on single-photon emission computed tomography (SPECT) or PET. Clinical syndrome may be further supported by genetic or pathological confirmation.

With respect to nfvPPA, diagnosis typically requires either agrammatism in language production or effortful, halting speech with inconsistent speech sound errors and distortions (AOS), along with two of the three remaining core features (impaired comprehension of syntactically complex sentences, spared single-word comprehension and spared object knowledge). In addition, neuroimaging consistent with nfvPPA supports diagnosis, and typically shows either predominant left posterior fronto-insular atrophy on MRI, or predominant left posterior fronto-insular hypoperfusion or hypometabolism on SPECT or PET, or both. Clinical syndrome may be further supported by genetic or pathological confirmation.

With respect to svPPA, diagnosis typically requires both impaired confrontation naming, and single-word comprehension, with at least 3 out of 4 additional core features (impaired object knowledge, surface dyslexia or dysgraphia, spared repetition and spared speech production). In addition, neuroimaging consistent with svPPA supports diagnosis, and typically shows either predominant anterior temporal lobe atrophy, or predominant anterior temporal hypoperfusion or hypometabolism on SPECT or PET, or both. Clinical syndrome may be further supported by genetic or pathological confirmation.

As used herein “subject” refers to a human being at any age, but typically an adult e.g., between 45 to 65, who suffers from the pathology. Preferably, this term encompasses individuals who are at risk to develop the pathology. The subject may exhibit one or more symptoms associated with the pathology. Alternatively, the subject may have a genetic predisposition to FTD.

As used herein “micro RNA” abbreviated as “miRNA” or “miR” refers to a sequence in the family of non-coding single-stranded RNA molecules of about 19-28 nucleotides in length, which regulate gene expression (acting as post-transcriptional regulators). MicroRNAs are typically processed from pre-miR (pre-microRNA precursors, typically of 45-90, 60-80 or 60-70 nucleotides). Pre-miRs are a set of precursor miRNA molecules transcribed by RNA polymerase III that are efficiently processed into functional miRNAs (i.e. mature miRNAs). According to one embodiment, this term encompasses any type of micoRNA including 5 prime (i.e. miR or 5p) or 3 prime (i.e. miR* or 3p) and their precursors.

Exemplary miRNAs and their precursors are provided below (accession numbers per miRbase).

hsa-miR-361-5p, MIMAT0000703 (SEQ ID NO: 1)
hsa-miR-629-5p, MIMAT0004810 (SEQ ID NO: 2)
hsa-miR-628-3p, MIMAT0003297 (SEQ ID NO: 3)
hsa-miR-379-5p, MIMAT0000733 (SEQ ID NO: 4)
hsa-miR-1-3p, MIMAT0000416 (SEQ ID NO: 5)
hsa-miR-26a-5p, MIMAT0000082 (SEQ ID NO: 6)
hsa-miR-125a-5p, MIMAT0000443 (SEQ ID NO: 7)
hsa-miR-125b-5p, MIMAT0000423 (SEQ ID NO: 8)
hsa-miR-142-5p, MIMAT0000433 (SEQ ID NO: 9)
hsa-miR-340-5p, MIMAT0004692 (SEQ ID NO: 10)
hsa-miR-326, MIMAT0000756 (SEQ ID NO: 11)
hsa-miR-128-3p, MIMAT0000424 (SEQ ID NO: 12)
hsa-miR-423-5p, MIMAT0004748 (SEQ ID NO: 13)
hsa-miR-107, MIMAT0000104 (SEQ ID NO: 14)
hsa-miR-23b-3p, MIMAT0000418 (SEQ ID NO: 15)
hsa-miR-320c, MIMAT0005793 (SEQ ID NO: 16)
hsa-miR-1306-5p, MIMAT0022726 (SEQ ID NO: 17)
hsa-let-7d-3p, MIMAT0004484 (SEQ ID NO: 18)
hsa-miR-378a-3p, MIMAT0000732 (SEQ ID NO: 19)
hsa-miR-103a-3p, MIMAT0000101 (SEQ ID NO: 20)
hsa-miR-148a-3p, MIMAT0000243 (SEQ ID NO: 21)
hsa-miR-409-3p, MIMAT0001639 (SEQ ID NO: 22)
hsa-miR-148b-3p, MIMAT0000759 (SEQ ID NO: 23)

According to one embodiment, the term miRNA comprises a combination of any two or more (e.g. 2, 3, 4, 5 or more) of the above described miRNAs.

Thus, according to one embodiment, the at least one micro RNA (miR) does not exceed 100 miRs.

According to one embodiment, the at least one micro RNA (miR) does not exceed 50 miRs.

According to one embodiment, the at least one micro RNA (miR) does not exceed 30 miRs.

According to one embodiment, the at least one micro RNA (miR) does not exceed 25 miRs.

According to one embodiment, the at least one micro RNA (miR) does not exceed 24 miRs.

According to one embodiment, the at least one micro RNA (miR) does not exceed 23 miRs.

According to one embodiment, the at least one micro RNA (miR) does not exceed 22 miRs.

According to one embodiment, the at least one micro RNA (miR) does not exceed 21 miRs.

According to one embodiment, the at least one micro RNA (miR) does not exceed 20 miRs.

According to one embodiment, the at least one micro RNA (miR) does not exceed 19 miRs.

According to one embodiment, the at least one micro RNA (miR) does not exceed 18 miRs.

According to one embodiment, the at least one micro RNA (miR) does not exceed 17 miRs.

According to one embodiment, the at least one micro RNA (miR) does not exceed 16 miRs.

According to one embodiment, the at least one micro RNA (miR) does not exceed 15 miRs.

According to one embodiment, the at least one micro RNA (miR) does not exceed 14 miRs.

According to one embodiment, the at least one micro RNA (miR) does not exceed 13 miRs.

According to one embodiment, the at least one micro RNA (miR) does not exceed 12 miRs.

According to one embodiment, the at least one micro RNA (miR) does not exceed 11 miRs.

According to one embodiment, the at least one micro RNA (miR) does not exceed 10 miRs.

According to one embodiment, the at least one micro RNA (miR) does not exceed 9 miRs.

According to one embodiment, the at least one micro RNA (miR) does not exceed 8 miRs.

According to one embodiment, the at least one micro RNA (miR) does not exceed 7 miRs.

According to one embodiment, the at least one micro RNA (miR) does not exceed 6 miRs.

According to one embodiment, the at least one micro RNA (miR) does not exceed 5 miRs.

According to one embodiment, the at least one micro RNA (miR) comprise 2-25 miRs.

According to one embodiment, the at least one micro RNA (miR) comprise 2-24 miRs.

According to one embodiment, the at least one micro RNA (miR) comprise 2-23 miRs.

According to one embodiment, the at least one micro RNA (miR) comprise 2-22 miRs.

According to one embodiment, the at least one micro RNA (miR) comprise 2-22 miRs.

According to one embodiment, the at least one micro RNA (miR) comprise 2-21 miRs.

According to one embodiment, the at least one micro RNA (miR) comprise 2-20 miRs.

According to one embodiment, the at least one micro RNA (miR) comprise 5-25 miRs.

According to one embodiment, the at least one micro RNA (miR) comprise 5-24 miRs.

According to one embodiment, the at least one micro RNA (miR) comprise 5-23 miRs.

According to one embodiment, the at least one micro RNA (miR) comprise 5-22 miRs.

According to one embodiment, the at least one micro RNA (miR) comprise 5-22 miRs.

According to one embodiment, the at least one micro RNA (miR) comprise 5-21 miRs.

According to one embodiment, the at least one micro RNA (miR) comprise 5-20 miRs.

According to one embodiment, the at least one micro RNA (miR) comprise 10-25 miRs.

According to one embodiment, the at least one micro RNA (miR) comprise 10-24 miRs.

According to one embodiment, the at least one micro RNA (miR) comprise 10-23 miRs.

According to one embodiment, the at least one micro RNA (miR) comprise 10-22 miRs.

According to one embodiment, the at least one micro RNA (miR) comprise 10-22 miRs.

According to one embodiment, the at least one micro RNA (miR) comprise 10-21 miRs.

According to one embodiment, the at least one micro RNA (miR) comprise 10-20 miRs.

According to one embodiment, the at least one miR comprises a range selected from the group consisting of 2-24, 2-20, 2-18, 2-16, 2-14, 2-12, 2-10, 2-8, 2-6 and 2-4.

According to a specific embodiment, the present teachings contemplate detecting at least one additional micro RNA (miR) selected from the group consisting of hsa-miR-326, hsa-miR-128-3p, hsa-miR-423-5p, hsa-miR-107, hsa-miR-23b-3p, hsa-miR-320c, hsa-miR-1306-5p, hsa-let-7d-3p, hsa-miR-378a-3p, hsa-miR-103a-3p, hsa-miR-148a-3p, hsa-miR-409-3p and hsa-miR-148b-3p.

According to a specific embodiment, the at least one micro RNA (miR) comprise hsa-miR-361-5p and hsa-miR-629-5p.

According to a specific embodiment, the at least one micro RNA (miR) comprise hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p, hsa-miR-340-5p, hsa-miR-326, hsa-miR-128-3p, hsa-miR-423-5p, hsa-miR-107, hsa-miR-23b-3p, hsa-miR-320c, hsa-miR-1306-5p, hsa-let-7d-3p, hsa-miR-378a-3p, hsa-miR-103a-3p, hsa-miR-148a-3p, hsa-miR-409-3p and hsa-miR-148b-3p.

According to a specific embodiment, the at least one micro RNA (miR) comprise miR-1-3p, miR-361-5p, miR-629-5p, miR-423-5p.

According to a specific embodiment, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p and miR-23b-3p.

According to a specific embodiment, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p, miR-23b-3p, miR-326 and miR-103a-3p.

According to a specific embodiment, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p, miR-23b-3p, miR-326, miR-103a-3p, miR-142-5p, miR-125a-5p, miR-26a-5p, miR-148a-3p, miR-379-5p and miR-320c.

According to a specific embodiment, the at least one micro RNA (miR) comprise miR-1-3p, miR-361-5p, miR-629-5p, miR-423-5p, miR-148b-3p, miR-628-3p, miR-409-3p, miR-378a-3p, miR-125b-5p, miR-23b, miR-326, miR-103a-3p, miR-142-5p, miR-125a-5p, miR-26a-5p, miR-148a-3p, miR-379-5p, miR-320c, let-7d-3p, miR-340-5p and miR-1306-5p.

According to a specific embodiment, the at least one micro RNA (miR) is selected from the group consisting of hsa-miR-361-5p, hsa-miR-629-5p, hsa-miR-26a-5p, hsa-miR-23b-3p, hsa-miR-326, hsa-miR-103a-3p, hsa-miR-379-5p, hsa-miR-320c, hsa-miR-128-3p, hsa-miR-148a-3p, hsa-miR-628-3p, hsa-miR-423-5p and hsa-let-7d-3p.

According to a specific embodiment, the at least one micro RNA (miR) comprise hsa-miR-361-5p, hsa-miR-629-5p, hsa-miR-26a-5p, hsa-miR-23b-3p, hsa-miR-326, hsa-miR-103a-3p, hsa-miR-379-5p, hsa-miR-320c, hsa-miR-128-3p, hsa-miR-148a-3p, hsa-miR-628-3p, hsa-miR-423-5p and hsa-let-7d-3p.

Diagnosing FTD according to some embodiments of the invention is performed with an acceptable level of clinical or diagnostic accuracy. An “acceptable degree of diagnostic accuracy”, is herein defined as a test or assay (such as the test used in some aspects of the invention) in which the to AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods diagnose with at least 75% total accuracy, more preferably 80%, 85%, 90%, 95%, 97%, 98%, 99% or greater total accuracy. Alternatively, the methods predict the correct management or treatment with an MCC larger than 0.2, 0.3, 0.4, 0.5, 0.6, 0.7, 0.8, 0.9 or 1.0.

Alternatively, the methods diagnose with at least or about 88% total accuracy.

According to some embodiments, diagnosis is done of cell-free biological samples.

The biological sample may comprise non-cellular vesicles, i.e., cell derived components which are not intact cells. These may be exosomes.

As used herein “a biological sample” refers to a sample of fluid or tissue sample derived from a subject. Examples of fluid samples include, but are not limited to, blood, plasma, serum, cerebrospinal fluid (CSF), lymph fluid, tears, saliva, sputum, urine and semen. An example of a tissue sample includes a brain tissue sample or a nerve tissue sample (e.g. for post-mortem diagnosis).

According to a specific embodiment, the biological sample is cell-free.

According to a specific embodiment, the biological sample comprises cell-free miRNA.

Typically, a biological sample is obtained from a subject (e.g. plasma, blood or CSF) and cells are removed therefrom when needed. Cell-free samples include, but are not limited to, plasma, serum and CSF.

Procedures for retrieving biological samples (e.g., blood samples or CSF samples) from individuals are well known in the art. Such procedures include, but are not limited to, standard blood retrieval procedures, lumbar puncture or urine collection. These and other procedures for obtaining biological samples are described in details in www(dot)healthatoz(dot)com/healthatoz/Atoz/search(dot)asp.

Regardless of the procedure employed, once the biological sample is obtained, the level of miRNA (e.g. cell-free miRNA) in the biological sample is determined.

As used herein the phrase “cell-free miRNA” refers to miRNA present within the cell-free fraction of a biological sample. The cell-free miRNA described herein is not comprised in intact cells (i.e., comprising uncompromised plasma membrane) but may be associated with cell-derived vesicles (e.g. exosomes).

Cell-free miRNA may be extracted from the biological sample according to any method known in the art. For instance, after obtaining the biological sample (i.e. blood or CSF), all nucleated cells are removed from the sample by two centrifugation cycles (e.g. at 1,600×g for 10 minutes at 4° C.). Total RNA is extracted from the cell-free sample (e.g. plasma or serum or CSF) using, for example, the miRNeasy micro kit (Qiagen, Hilden, Germany) and quantified with, for example, Qubit fluorometer using RNA broad range (BR) assay kit (Thermo Fisher Scientific, Waltham, Mass.).

According to one embodiment, the method further comprises retrieving a biological sample from the subject.

According to one embodiment, two or more samples are collected from a subject.

According to one embodiment, a sample is obtained at disease onset.

A “control” or “control sample” may be of the subjecy before showing symptoms of the disease or of a healthy subject(s) who are affirmed as not having a neurodegenerative disease e.g., FTD.

Such control samples are typically obtained from subjects of the same age and gender. Furthermore, normal miRNA levels may be determined experimentally or derived from the literature if available.

The expression level of the miRNA in the biological samples of some embodiments of the invention can be determined using any methods known in the arts. For example, a number of miRNA quantitative analysis methods have been developed, including e.g. miRNA chip arrays, SYBR Green I-based miRNA qRT-PCR assays [discussed in Raymond et al., Simple, quantitative primer-extension PCR assay for direct monitoring of microRNAs and short-interfering RNAs. RNA (2005) 11], stem-loop-based TaqMan assays [discussed in Chen C et al., Real-time quantification of microRNAs by stem-loop RT-PCR. Nucleic Acids Res (2005) 33(20):e179], beads-based assays, high throughput sequencing and the like.

According to one embodiment, detecting a specific miRNA (e.g. cell-free miRNA) with or without a step of amplification typically involves the use of at least one of next generation sequencing (NGS), real time PCR, nCounter (Nanostring), or microarray (as described in detail in the ‘general materials and experimental procedures’ section of the Examples section which follows).

According to a specific embodiment, the level of any of the miRs tested is higher or lower by at least 1.5-fold, than that of the control sample, to reach a diagnosis.

TABLE 1

| Feature | Increase risk |
|---|---|
| mir-629 | high |
| mir-361-5p | high |
| miR-378a-3p | low |
| miR-326 | high |
| miR-340 | high |
| miR-125a-5p | low |
| miR-142 | high |
| mir-26 | low |
| miR-23b-3p | low |
| miR-379-5p | low |
| miR-125b-5p | low |
| miR-628-3p | high |
| mir-320c | high |
| miR-1306-5p | high |
| miR-107 | low |
| miR-1-3p | low |
| mir-128-3p | mixed |
| let-7d-3p | mixed |

Mixed-indicates higher or lower compared to healthy which varies in different subjects.

Once diagnosis is made it can be corroborated by the use of Gold standard assays.

These include, but are not limited to, obstructive sleep apnea (OSA) test, as executive dysfunction and behavior changes are common in OSA. If the classic features of OSA are present (e.g., loud disruptive snoring, snorts and apneic pauses while sleeping, crowded oropharynx, excessive daytime sleepiness, repetitive desaturations on overnight oximetry), then referral to a sleep medicine specialist and polysomnography is indicated.

Blood work should be done to exclude alternative causes of cognitive symptoms, including a basic metabolic panel, CBC, RPR, ESR, B12 level and thyroid studies. Vascular risk factors can be assessed. Infections (including HIV), immune-based dementias and neoplastic/paraneoplastic etiologies are occasionally causative or significant contributors, and should be considered.

When a family history is positive, genetic testing of the diagnosed patient can be undertaken.

A full neuropsychological testing evaluation can be used to better assess the pattern of cognitive loss in an individual suspected of having FTD and to help rule out psychiatric etiologies for an individual's symptoms. Screening neuropsychological testing takes several hours and is done by a neuropsychologist (or sometimes under direction of a neuropsychology technician). It provides additional supportive evidence for the FTD diagnosis, keeping in mind that some patients perform within normal limits when features are mild. When PPA is suspected, a comprehensive evaluation by a speech/language pathologist can be performed.

Brain imaging is indicated in all individuals with symptoms of FTD to rule out structural causes. MRI scanning will identify small vessel ischemia, subdural hematomas, strategically placed tumors and hydrocephalus. Additionally, the pattern of brain atrophy can support the diagnosis. Severe “knife-edge atrophy” of the frontal and/or anterior temporal lobes may be seen. Often this is asymmetrical. There is often relative sparing of the posterior head regions.

The MRI is more sensitive for assessing vascular changes and subtle patterns of atrophy, but it requires an individual to lie still for 15 to 30 minutes. If the subject is unable to tolerate this, or if they are severely claustrophobic, a CT scan may be more realistic. If the MRI or CT scan does not show atrophy, and the diagnosis remains unclear, a fluorodeoxyglucose positron emission tomography (FDG-PET) scan or SPECT (single proton emission CT) scan may be considered. FDG-PET scans are more specific.

Lumbar puncture is another test that can be used to rule out mimicking conditions (infection, immune etiologies, carcinomatous and paraneoplastic syndromes). Measurement of CSF phospho-tau, total tau and Beta-amyloid can sometimes support the diagnosis of FTD over Alzheimer's disease. As this is an invasive procedure, the value of additional information to be gained should be discussed with patient and family.

Electrophysiologic testing is sometimes warranted in patients with possible FTD. The pattern of change in electroencephalography is nonspecific in FTD; often the test is normal. It may be used to rule out nonepileptic seizures and other systemic (hyperammonemia) or infectious (prion) disorders. Although nonspecific, this testing is easily obtained at many hospitals, is less costly, and it is relatively noninvasive

The methods of some embodiments of the invention can be corroborated by one, two or more of the above tests.

According to some embodiments of the invention, the method further comprises informing the subject of the diagnosis.

As used herein the phrase “informing the subject” refers to advising the subject that based on the methods of some embodiments of the invention the subject should seek a suitable treatment regimen.

Once diagnosis is made, treatment can be selected/designed according to the finding. i.e., FTD or its sub-classification.

Thus, according to an aspect of the invention there is provided a method of treating Frontotemporal dementia (FTD) in a subject in need thereof, the method comprising:

(a) diagnosing FTD as described herein;

(b) treating the subject with a drug which ameliorates symptoms associated with FTD.

According to an alternative embodiment, there is provided a method of treating Frontotemporal dementia (FTD) in a subject in need thereof, the method comprising: treating the subject with a drug which ameliorates symptoms associated with FTD, wherein the subject has been diagnosed with FTD as described herein.

According to an alternative embodiment, there is provided a method of selecting a treatment for a subject in need thereof, the method comprising:

(a) diagnosing FTD as described herein;

(b) selecting treatment for the subject with a drug which ameliorates symptoms associated with FTD.

As used herein, the terms “treating” or “treatment” include abrogating, substantially inhibiting, slowing or reversing the progression of a condition, substantially ameliorating clinical symptoms or substantially preventing the appearance of clinical symptoms of FTD.

Any drug or medicament for the treatment of FTD may be used in accordance with the present teachings.

According to one embodiment, the drug or medicament is a candidate drug or approved drug/treatment (e.g. an experimental drug or a drug in a clinical trial).

Exemplary agents which may be used in accordance with the present teachings for the treatment of FTD or FTD symptoms include, but are not limited to, drugs which are used to manage the behavioral symptoms, antidepressants, drugs for treatment of aggression, agitation and psychosis, and drugs for the treatment of dementia. Exemplary drugs for the treatment of FTD include, but are not limited to, selective serotonin reuptake inhibitors (SSRIs), anti-depressants (e.g. trazodone), neuroleptics/a (e.g. olanzapine, risperidone and aripiprazole), cholinergic agents (e.g. rivastigmine), acetylcholinesterase inhibitors (e.g. galantamine), NMDA receptor antagonists (e.g. Memantine). Additionally, gene therapy, antisense oligonucleotide therapy, and cellular base therapy (e.g. injection of mesenchymal stem cells) can be used for the treatment of FTD.

According to one embodiment, the subject is treated with a nutraceutical composition i.e. any substance that may be considered a food or part of a food and provides medical or health benefits, including the prevention and treatment of disease. In some embodiments, a nutraceutical composition is intended to supplement the diet and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

According to one embodiment, the subject is treated with physical therapy, or any other therapy which may assist muscle movement or pain.

According to one embodiment, the subject is treated with an assistive device. Any assistive device can be used according to the present teachings including, but not limited to, a cane, a leg brace, a hand and/or wrist splint, a wheelchair (such as a power wheelchair), a communication device, and a mechanical lift. Additionally or alternatively, any of a feeding tube, a urinary catheter, a ventilator (e.g., noninvasive such as a BiPAP e.g. by Philips Respironics) or invasive ventilator (e.g. via tracheostomy) or a pacemaker may be used.

Any of the above described agents may be administered or used individually or in combination.

The methods of some embodiments of the invention may be further used for selecting subjects for enrollment in a clinical trial involving treatment of FTD.

Thus, according to an aspect of the invention there is provided a method of selecting subjects for enrollment in a clinical trial involving treatment of Frontotemporal dementia (FTD), the method comprising:

(a) determining diagnosis of a subject as described herein; and

(c) identifying the subject as being suitable for said clinical trial based on the criteria of said clinical trial.

According to a specific embodiment, the subject is a carrier of a pathogenic GRN (progranulin gene) mutation.

According to a specific embodiment, the subject displays FTD symptoms.

This aspect allows early enrollment in clinical trials for better efficacy of the tested drug/treatment.

It is expected that during the life of a patent maturing from this application many relevant drugs or medicaments for the treatment of FTD will be developed and the scope of the term drug or medicament for the treatment of FTD is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Standard Protocol Approvals, Registrations, and Patient Consents

Approvals were obtained from the local research ethics committee and all participants provided written consent (or gave verbal permission for a carer to sign on their behalf).

### Study Design

Based on power analysis, it was found that about 20 control and 50 FTD samples are required to obtain an ROC of 0.7 with a power of 80% and a p-value of 0.05. We determined the sample size based on these calculations. Because sample processing was done in different batches, samples were randomly allocated to the batches and within each batch, the number of control and FTD samples was balanced in order to reduce batch-associated bias.

### Participants and Sampling

Participants were enrolled in the longitudinal FTD cohort studies at UCL. Frozen plasma samples from the UCL FTD Biobank were shipped to the Weizmann Institute of Science for molecular analysis. Study cohort I: 52 FTD patients, 21 healthy controls. Study cohort II: 117 FTD patients, 35 healthy controls. FTD patients were further assigned into two groups with predicted pathology of TDP-43 or tau, based on genetics and clinical phenotype. Patients positive for C9ORF72 repeats and progranulin (PRGN) mutations and/or presented with semantic dementia, were predicted to have TDP-43 pathology, while patients with MAPT mutations were predicted to have tau pathology. Demographic data are detailed in Table 2, hereinbelow.

TABLE 2
Summary of demographic and clinical characteristics
of FTD Cohorts I and II and control samples.

| | Control | FTD |
|---|---|---|
| Number of subjects (% males) | 125 (31%) | 168 (65%) |
| Age at enrolment | 59.5 ± 10.2 | 65.8 ± 8.1 yr. |
| Age of onset (1^{st} reported | N/A | 60.3 ± 8.3 yr. |
| symptoms) | | |
| Disease duration at enrolment | N/A | 5.5 ± 3.4 yr |
| Clinical subtype | N/A | 81/40/28/4/15 |
| (bvFTD/PNFA/SD/FTD-ALS/others) | | |
| Mutation carriers | N/A | 18/13/13/2 |
| (C9ORF72/MAPT/GRN/TBK1) | | |
| Likely pathology (TDP-43/Tau) | N/A | 63/18 |

bvFTD: behavioural FTD; PNFA: progressive nonfluent aphasia; SD: semantic dementia.
Mean ± SD.

Healthy controls were typically spouses or relatives of patients not inflicted with neurodegenerative disorder. Demographic data are detailed in Table 2, hereinabove. Plasma samples were stored in −80° C. until RNA extraction and subsequent small RNA next generation sequencing.

### Small RNA Next Generation Sequencing

Total RNA was extracted from plasma using the miRNeasy micro kit (Qiagen, Hilden, Germany) and quantified with Qubit fluorometer using RNA broad range (BR) assay kit (Thermo Fisher Scientific, Waltham, Mass.). For small RNA next generation sequencing (NGS), libraries were prepared from 7.5 ng of total RNA using the QIAseq miRNA Library Kit and QIAseq miRNA NGS 48 Index IL (Qiagen), by an experimenter who was blinded to the identity of samples. Following 3′ and 5′ adapter ligation, small RNA was reverse transcribed, using unique molecular identifier (UMI), primers of random 12-nucleotide sequences. This way, precise linear quantification miRNA is achieved, overcoming potential PCR-induced biases (18). cDNA libraries were amplified by PCR for 22 cycles, with a 3′ primer that includes a 6-nucleotide unique index. Following size selection and cleaning of libraries with magnetic beads, quality control was performed by measuring library concentration with Qubit fluorometer using dsDNA high sensitivity (HS) assay kit (Thermo Fisher Scientific, Waltham, Mass.) and confirming library size with Tapestation D1000 (Agilent). Libraries with different indices were multiplexed and sequenced on a single NextSeq 500/550 v2 flow cell (Illumina), with 75bp single read and 6bp index read. Fastq files were demultiplexed using the User-friendly Transcriptome Analysis Pipeline (UTAP) developed at the Weizmann Institute (33). Sequences were mapped to the human genome using Qiagen GeneGlobe analysis web tool, which uses miRBase V21 as a reference.

### Statistical Analysis and Machine Learning

miRNA NGS data was analyzed via DESeq2 package in R Project for Statistical Computing (34, 35).

The FTD-disease binary classifier was developed using Gradient Boosting Classifier, a machine learning algorithm that uses a gradient boosting framework. Gradient Boosting trees [Elith and Hastie, 2008, Witten et al., 2017], a decision-tree-based ensemble model, differ fundamentally from conventional statistical techniques that aim to fit a single model using the entire dataset. Such ensemble approach improves performance by combining strengths of models that learn the data by recursive binary splits, such as trees, and of “boosting”, an adaptive method for combining several simple (base) models. At each iteration of the gradient boosting algorithm, a subsample of the training data is selected at random (without replacement) from the entire training data set, and then a simple base learner is fitted on each subsample. The final boosted trees model is an additive tree model, constructed by sequentially fitting such base learners on different subsamples. This procedure incorporates randomization, which is known to substantially improve the predictor accuracy and also increase robustness. Additionally, boosted trees can fit complex nonlinear relationships, and automatically handle interaction effects between predictors as addition to other advantages of tree-based methods, such as handling features of different types and accommodating missing data. Hence, in many cases their predictive performance is superior to most traditional modelling methods.

Additional gain of these algorithms is the various loss functions that can be applied, both for binary and multi-class problems. Using the softmax loss function, we explicitly estimated the class conditional probabilities, which allow us to demonstrate the performance of each of the classifiers both as “soft-classifiers” (i.e., predicting class probabilities) and “hard-classifiers” (i.e., setting a probability threshold and predicting a class). The former approximates a continuous number as output—the class conditional probabilities—and then performs classification based on these estimated probabilities. In contrast, hard classifiers output a discrete number as the decision -directly targeting the classification decision boundary, without producing the probability estimation.

A gradient boosting classifier was developed with a feature set of 132 miRNA predictors, age and sex. Dataset was partitioned to training-set (75%) and validation-set (25%) which was used as held-out data. The training-set was cross validated during training with stratified 3-fold cross validation. An ROC was generated for each of the folds and individual and mean AUCs were calculated along with 95% confidence intervals.

The chosen hyperparams in FTD-disease classifier: ccp_alpha=0.0, learning_rate=0.5, max_depth=8, max_features=0.45, min_samples_leaf=14, min_samples_split=8, n_estimators=100, subsample=0.45 and tol=0.0001.

### Predictor Selection by Recursive Feature Elimination (RFE)

For selecting the most predictive features in the meta-cohort, the present inventors used the cross validated Recursive Feature Elimination (RFE) algorithm, an efficient recursive approach for eliminating features from a training dataset with K-fold cross validation. The chosen classifier used for RFE was Extra Trees Classifier with following hyperparms: criterion=“entropy”, max_features=0.9, n_estimators=10. The performance scorer used to optimize selection: weighted ROC AUC score.

### Feature Importance and SHAP Analysis

Although gradient boosting trees are complex models, they automatically provide an approximation of feature importance from the trained boosted trees. A miRNA predictor is assigned with an importance score in every single tree, where the Gini purity index is used to assess split points in the tree. The score of a feature is calculated based on the amount of improvement in the Gini index achieved by split points that include the feature, weighted by the number of observations in that node. The final importance score of a feature is calculated by an average across all decision trees within the final model. Importance scores of 132 miRNAs were used here in order to rank features in the multi-disease classifier, and thus reduce the dimension of miRNA measurements needed for prediction by 69% (41 features in total).

For local interpretability of the predictive model, the present inventors used SHapley Additive exPlanations (SHAP), the current state of the art in Machine Learning explainability tools. Giving the smallest set of features that reduced the loss function the most (by XGBoost feature importance), SHAP provides estimates and visualizations to infer what decisions the model is making. This is achieved by quantifying the contribution that each feature brings to each prediction made by the model.

### Example 1

### A Plasma miRNA Classifier for FTD

In order to characterize the potential of plasma miRNAs as biomarkers for FTD, a cohort of n=168 plasma samples of FTD patients and n=125 plasma samples of healthy controls (subject information in Table 2) was assessed. RNA was purified and next generation sequencing (NGS) performed. As many as 1838 individual miRNA species were aligned to the human miRNA repository (miRBase V21).

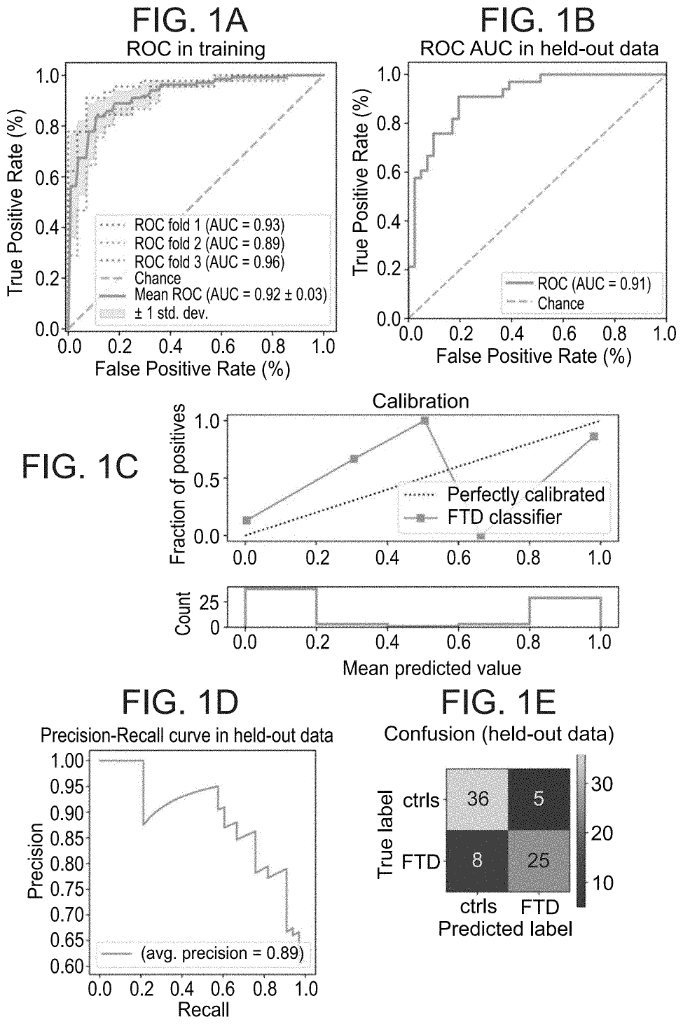

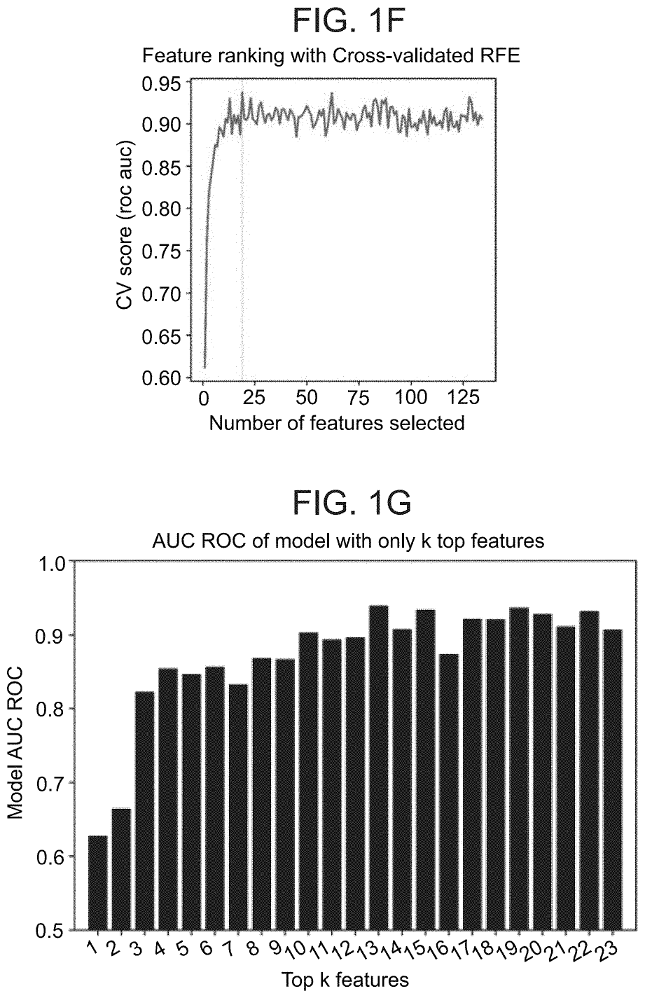

Only 132 miRNA species exceeded a quality control threshold (cut-off of ≥100 UMIs per sample averaged on all samples). A disease-prediction model for FTD diagnosis was developed, using data from a cohort of n=293 subjects with 132 miRNA measurements, age and sex (total of 134 features). Machine learning algorithm was used for model development on ¾ of the data with 3-fold stratified cross validation between different data folds (K=3×¼ of data). Importantly, 25% of the data (74 subjects, 41 FTD and 33 controls) was completely held-out for bona fide validation of the model on previously-unseen data [FIG. 1A-B]. It was possible to further reduce the number of features from 134 to just 23 most predictive features, while keeping fine classifying accuracy (FIG. 1F, FIG. 1G, listed hereinbelow).

Of the list:

hsa-miR-361-5p, MIMAT0000703 (SEQ ID NO: 1)
hsa-miR-629-5p, MIMAT0004810 (SEQ ID NO: 2)
hsa-miR-628-3p, MIMAT0003297 (SEQ ID NO: 3)
hsa-miR-379-5p, MIMAT0000733 (SEQ ID NO: 4)
hsa-miR-1-3p, MIMAT0000416 (SEQ ID NO: 5)
hsa-miR-26a-5p, MIMAT0000082 (SEQ ID NO: 6)
hsa-miR-125a-5p, MIMAT0000443 (SEQ ID NO: 7)
hsa-miR-125b-5p, MIMAT0000423 (SEQ ID NO: 8)
hsa-miR-142-5p, MIMAT0000433 (SEQ ID NO: 9)
hsa-miR-340-5p, MIMAT0004692 (SEQ ID NO: 10)
hsa-miR-326, MIMAT0000756 (SEQ ID NO: 11)
hsa-miR-128-3p, MIMAT0000424 (SEQ ID NO: 12)
hsa-miR-423-5p, MIMAT0004748 (SEQ ID NO: 13)
hsa-miR-107, MIMAT0000104 (SEQ ID NO: 14)
hsa-miR-23b-3p, MIMAT0000418 (SEQ ID NO: 15)
hsa-miR-320c, MIMAT0005793 (SEQ ID NO: 16)
hsa-miR-1306-5p, MIMAT0022726 (SEQ ID NO: 17)
hsa-let-7d-3p, MIMAT0004484 (SEQ ID NO: 18)
hsa-miR-378a-3p, MIMAT0000732 (SEQ ID NO: 19)
hsa-miR-103a-3p, MIMAT0000101 (SEQ ID NO: 20)
hsa-miR-148a-3p, MIMAT0000243 (SEQ ID NO: 21)
hsa-miR-409-3p, MIMAT0001639 (SEQ ID NO: 22)
hsa-miR-148b-3p, MIMAT0000759 (SEQ ID NO: 23)

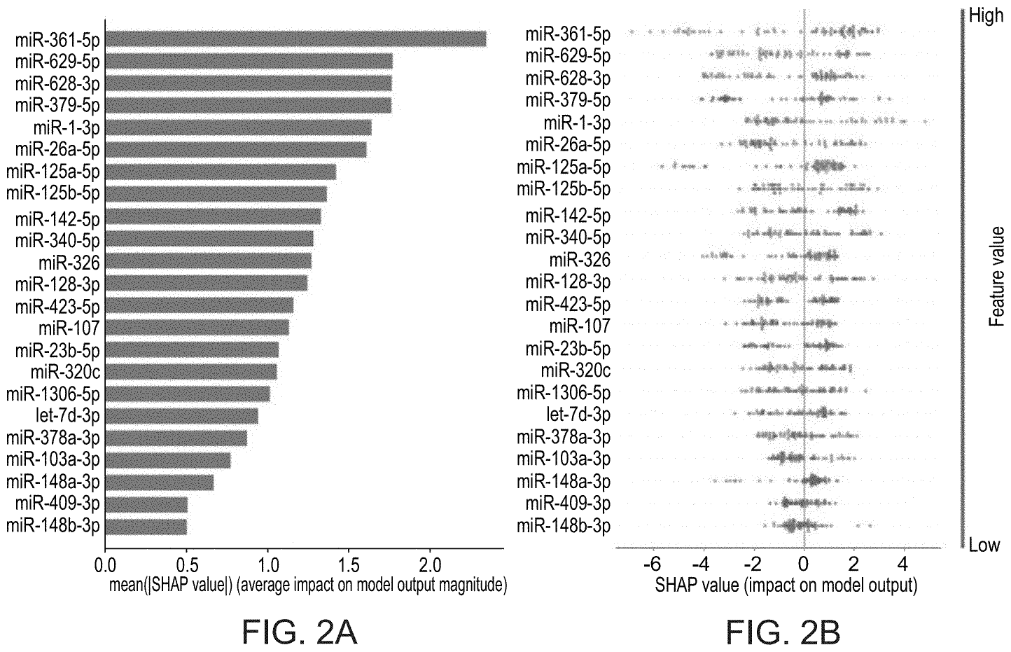

The best FTD classifier presented a prediction capacity of 93% [(mean ROC AUC of 0.93 during training (FIG. 1A)], and kept being exceptionally informative on previously-unseen data with prediction capacity of 91%. (ROC AUC of 0.91 on the held-out validation set). Post-hoc analysis of the FTD classifier breaks down the contribution of individual features and reveals miRNA predictors of high disease risk (FIG. 2A-B).

The present inventors predicted FTD with several models, increasing in number from 1-feature to 21-miRNA model. Each model contains the miRNAs in the previous list and additional miRNAs.

The prediction accuracy is measured as area under the curve (AUC) of the receiver operating characteristic (ROC) curve. Maximum possible value of AUC is 1.

Following are the models, with their AUC values:

Model with 1 miRNA (miR-1-3p): AUC=0.627
Model with 4 miRNAs (miR-1-3p, miR-361-5p, miR-629-5p, miR-423-5p,): AUC=0.854
Model with 10 miRNAs (miR-1-3p, miR-361-5p, miR-629-5p, miR-423-5p, miR-148b-3p, miR-628-3p, miR-409-3p, miR-378a-3p, miR-125b-5p and miR-23b-3p : AUC=0.903.
Model with 12 miRNAs (miR-1-3p, miR-361-5p, miR-629-5p, miR-423-5p, miR-148b-3p, miR-628-3p, miR-409-3p, miR-378a-3p, miR-125b-5p, miR-23b-3p, miR-326 and miR-103a-3p): AUC=0.896
Model with 18 miRNAs (miR-1-3p, miR-361-5p, miR-629-5p, miR-423-5p, miR-148b-3p, miR-628-3p, miR-409-3p, miR-378a-3p, miR-125b-5p, miR-23b, miR-326, miR-103a-3p, miR-142-5p, miR-125a-5p, miR-26a-5p, miR-148a-3p, miR-379-5p, miR-320c): AUC=0.921
Model with 21 miRNAs (miR-1-3p, miR-361-5p, miR-629-5p, miR-423-5p, miR-148b-3p, miR-628-3p, miR-409-3p, miR-378a-3p, miR-125b-5p, miR-23b, miR-326, miR-103a-3p, miR-142-5p, miR-125a-5p, miR-26a-5p, miR-148a-3p, miR-379-5p, miR-320c, let-7d-3p, miR-340-5p, miR-1306-5p): AUC=0.91

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (other references are cited in the document)

1. R. J. Harvey, M. Skelton-Robinson, M. N. Rossor, The prevalence and causes of dementia in people under the age of 65 years. J Neurol Neurosurg Psychiatry 74, 1206-1209 (2003).

2. G. D. Rabinovici, B. L. Miller, Frontotemporal lobar degeneration: epidemiology, pathophysiology, diagnosis and management. CNS Drugs 24, 375-398 (2010).

3. J. S. Snowden, D. Neary, D. M. Mann, Frontotemporal dementia. Br J Psychiatry 180, 140-143 (2002).

4. G. Lee, C. J. Leugers, Tau and tauopathies. Prog Mol Biol Transl Sci 107, 263-293 (2012).

5. M. Neumann, D. M. Sampathu, L. K. Kwong, A. C. Truax, M. C. Micsenyi, T. T. Chou, J. Bruce, T. Schuck, M. Grossman, C. M. Clark, L. F. McCluskey, B. L. Miller, E. Masliah, I. R. Mackenzie, H. Feldman, W. Feiden, H. A. Kretzschmar, J. Q. Trojanowski, V. M. Lee, Ubiquitinated TDP-43 in frontotemporal lobar degeneration and amyotrophic lateral sclerosis. Science 314, 130-133 (2006).

6. M. Neumann, R. Rademakers, S. Roeber, M. Baker, H. A. Kretzschmar, I. R. Mackenzie, A new subtype of frontotemporal lobar degeneration with FUS pathology. Brain 132, 2922-2931 (2009).

7. M. Neumann, S. Roeber, H. A. Kretzschmar, R. Rademakers, M. Baker, I. R. Mackenzie, Abundant FUS-immunoreactive pathology in neuronal intermediate filament inclusion disease. Acta Neuropathol 118, 605-616 (2009).

8. F. Fontana, K. Siva, M. A. Denti, A network of RNA and protein interactions in Fronto Temporal Dementia. Front Mol Neurosci 8, 9 (2015).

9. A. Sieben, T. Van Langenhove, S. Engelborghs, J. J. Martin, P. Boon, P. Cras, P. P. De Deyn, P. Santens, C. Van Broeckhoven, M. Cruts, The genetics and neuropathology of frontotemporal lobar degeneration. Acta Neuropathol 124, 353-372 (2012).

10. J. van Swieten, M. G. Spillantini, Hereditary frontotemporal dementia caused by Tau gene mutations. Brain Pathol 17, 63-73 (2007).

11. C. Pottier, K. F. Bieniek, N. Finch, M. van de Vorst, M. Baker, R. Perkersen, P. Brown, T. Ravenscroft, M. van Blitterswijk, A. M. Nicholson, M. DeTure, D. S. Knopman, K. A. Josephs, J. E. Parisi, R. C. Petersen, K. B. Boylan, B. F. Boeve, N. R. Graff-Radford, J. A. Veltman, C. Gilissen, M. E. Murray, D. W. Dickson, R. Rademakers, Whole-genome sequencing reveals important role for TBK1 and OPTN mutations in frontotemporal lobar degeneration without motor neuron disease. Acta Neuropathol 130, 77-92 (2015).

12. E. Majounie, A. E. Renton, K. Mok, E. G. Dopper, A. Waite, S. Rollinson, A. Chio, G. Restagno, N. Nicolaou, J. Simon-Sanchez, J. C. van Swieten, Y. Abramzon, J. O. Johnson, M. Sendtner, R. Pamphlett, R. W. Orrell, S. Mead, K. C. Sidle, H. Houlden, J. D. Rohrer, K. E. Morrison, H. Pall, K. Talbot, O. Ansorge, A. L. S. F. T. D. C. Chromosome, F. F. A. French research network on, I. Consortium, D. G. Hernandez, S. Arepalli, M. Sabatelli, G. Mora, M. Corbo, F. Giannini, A. Calvo, E. Englund, G. Borghero, G. L. Floris, A. M. Remes, H. Laaksovirta, L. McCluskey, J. Q. Trojanowski, V. M. Van Deerlin, G. D. Schellenberg, M. A. Nalls, V. E. Drory, C. S. Lu, T. H. Yeh, H. Ishiura, Y. Takahashi, S. Tsuji, I. Le Ber, A. Brice, C. Drepper, N. Williams, J. Kirby, P. Shaw, J. Hardy, P. J. Tienari, P. Heutink, H. R. Morris, S. Pickering-Brown, B. J. Traynor, Frequency of the C9orf72 hexanucleotide repeat expansion in patients with amyotrophic lateral sclerosis and frontotemporal dementia: a cross-sectional study. Lancet Neurol 11, 323-330 (2012).

13. A. E. Renton, E. Majounie, A. Waite, J. Simon-Sanchez, S. Rollinson, J. R. Gibbs, J. C. Schymick, H. Laaksovirta, J. C. van Swieten, L. Myllykangas, H. Kalimo, A. Paetau, Y. Abramzon, A. M. Remes, A. Kaganovich, S. W. Scholz, J. Duckworth, J. Ding, D. W. Harmer, D. G. Hernandez, J. O. Johnson, K. Mok, M. Ryten, D. Trabzuni, R. J. Guerreiro, R. W. Orrell, J. Neal, A. Murray, J. Pearson, I. E. Jansen, D. Sondervan, H. Seelaar, D. Blake, K. Young, N. Halliwell, J. B. Callister, G. Toulson, A. Richardson, A. Gerhard, J. Snowden, D. Mann, D. Neary, M. A. Nalls, T. Peuralinna, L. Jansson, V. M. Isoviita, A. L. Kaivorinne, M. Holtta-Vuori, E. Ikonen, R. Sulkava, M. Benatar, J. Wuu, A. Chio, G. Restagno, G. Borghero, M. Sabatelli, I. Consortium, D. Heckerman, E. Rogaeva, L. Zinman, J. D. Rothstein, M. Sendtner, C. Drepper, E. E. Eichler, C. Alkan, Z. Abdullaev, S. D. Pack, A. Dutra, E. Pak, J. Hardy, A. Singleton, N. M. Williams, P. Heutink, S. Pickering-Brown, H. R. Morris, P. J. Tienari, B. J. Traynor, A hexanucleotide repeat expansion in C9ORF72 is the cause of chromosome 9p21-linked ALS-FTD. Neuron 72, 257-268 (2011).

14. M. A. van Es, O. Hardiman, A. Chio, A. Al-Chalabi, R. J. Pasterkamp, J. H. Veldink, L. H. van den Berg, Amyotrophic lateral sclerosis. Lancet 390, 2084-2098 (2017).

15. E. Feneberg, E. Gray, O. Ansorge, K. Talbot, M. R. Turner, Towards a TDP-43-Based Biomarker for ALS and FTLD. Mol Neurobiol 55, 7789-7801 (2018).

16. L. H. H. Meeter, E. G. Vijverberg, M. Del Campo, A. J. M. Rozemuller, L. Donker Kaat, F. J. de Jong, W. M. van der Flier, C. E. Teunissen, J. C. van Swieten, Y. A. L. Pijnenburg, Clinical value of neurofilament and phospho-tau/tau ratio in the frontotemporal dementia spectrum. Neurology 90, e1231-e1239 (2018).

17. T. Skillback, N. Mattsson, K. Blennow, H. Zetterberg, Cerebrospinal fluid neurofilament light concentration in motor neuron disease and frontotemporal dementia predicts survival. Amyotroph Lateral Scler Frontotemporal Degener 18, 397-403 (2017).

18. A. M. L. Coenen-Stass, I. Magen, T. Brooks, I. Z. Ben-Dov, L. Greensmith, E. Hornstein, P. Fratta, Evaluation of methodologies for microRNA biomarker detection by next generation sequencing. RNA Biol 15, 1133-1145 (2018).

19. C. Eitan, E. Hornstein, Vulnerability of microRNA biogenesis in FTD-ALS. Brain Res, (2016).

20. I. Magen, A. Coenen-Stass, A. Malaspina, L. Greensmith, C. H. Lu, P. Fratta, E. Hornstein, Circulating miRNAs as prognostic and longitudinal markers for amyotrophic lateral sclerosis. (In preparation).

21. K. S. Sheinerman, J. B. Toledo, V. G. Tsivinsky, D. Irwin, M. Grossman, D. Weintraub, H. I. Hurtig, A. Chen-Plotkin, D. A. Wolk, L. F. McCluskey, L. B. Elman, J. Q. Trojanowski, S. R. Umansky, Circulating brain-enriched microRNAs as novel biomarkers for detection and differentiation of neurodegenerative diseases. Alzheimers Res Ther 9, 89 (2017).

22. M. Grasso, P. Piscopo, G. Talarico, L. Ricci, A. Crestini, G. Tosto, M. Gasparini, G. Bruno, M. A. Denti, A. Confaloni, Plasma microRNA profiling distinguishes patients with frontotemporal dementia from healthy subjects. Neurobiol Aging, (2019).

23. J. Denk, F. Oberhauser, J. Kornhuber, J. Wiltfang, K. Fassbender, M. L. Schroeter, A. E. Volk, J. Diehl-Schmid, J. Prudlo, A. Danek, B. Landwehrmeyer, M. Lauer, M. Otto, H. Jahn, F. T. s. group, Specific serum and CSF microRNA profiles distinguish sporadic behavioural variant of frontotemporal dementia compared with Alzheimer patients and cognitively healthy controls. PLoS One 13, e0197329 (2018).

24. R. Schneider, P. McKeever, T. Kim, C. Graff, J. C. van Swieten, A. Karydas, A. Boxer, H. Rosen, B. L. Miller, R. Laforce, Jr., D. Galimberti, M. Masellis, B. Borroni, Z. Zhang, L. Zinman, J. D. Rohrer, M. C. Tartaglia, J. Robertson, F. T. D. I. Genetic, Downregulation of exosomal miR-204-5p and miR-632 as a biomarker for FTD: a GENFI study. J Neurol Neurosurg Psychiatry 89, 851-858 (2018).

25. S. Al-Hameed, M. Benaissa, H. Christensen, B. Mirheidari, D. Blackburn, M. Reuber, A new diagnostic approach for the identification of patients with neurodegenerative cognitive complaints. PLoS One 14, e0217388 (2019).

26. S. Baek, C. A. Tsai, J. J. Chen, Development of biomarker classifiers from high-dimensional data. Brief Bioinform 10, 537-546 (2009).

27. H. M. de Andrade, M. de Albuquerque, S. H. Avansini, S. R. C. de, D. B. Dogini, A. Nucci, B. Carvalho, I. Lopes-Cendes, M. C. Franca, Jr., MicroRNAs-424 and 206 are potential prognostic markers in spinal onset amyotrophic lateral sclerosis. J Neurol Sci 368, 19-24 (2016).

28. E. Tasca, V. Pegoraro, A. Merico, C. Angelini, Circulating microRNAs as biomarkers of muscle differentiation and atrophy in ALS. Clin Neuropathol 35, 22-30 (2016).

29. J. M. Toivonen, R. Manzano, S. Olivan, P. Zaragoza, A. Garcia-Redondo, R. Osta, MicroRNA-206: a potential circulating biomarker candidate for amyotrophic lateral sclerosis. PLoS One 9, e89065 (2014).

30. R. Waller, E. F. Goodall, M. Milo, J. Cooper-Knock, M. Da Costa, E. Hobson, M. Kazoka, H. Wollff, P. R. Heath, P. J. Shaw, J. Kirby, Serum miRNAs miR-206, 143-3p and 374b-5p as potential biomarkers for amyotrophic lateral sclerosis (ALS). Neurobiol Aging 55, 123-131 (2017).

31. J. R. Hodges, O. Piguet, Progress and Challenges in Frontotemporal Dementia Research: A 20-Year Review. J Alzheimers Dis 62, 1467-1480 (2018).

32. B. R. Brooks, R. G. Miller, M. Swash, T. L. Munsat, D. World Federation of Neurology Research Group on Motor Neuron, El Escorial revisited: revised criteria for the diagnosis of amyotrophic lateral sclerosis. Amyotroph Lateral Scler Other Motor Neuron Disord 1, 293-299 (2000).

33. R. Kohen, J. Barley, G. Hornung, G. Stelzer, E. Feldmesser, K. Kogan, M. Safran, D. Leshkowitz, UTAP: User-friendly Transcriptome Analysis Pipeline. BMC bioinformatics 24, 154 (2019).

34. R Core Team, R: A Language and Environment for Statistical Computing. Vienna, Austria. R Foundation for Statistical Computing (2015).

35. M. I. Love, W. Huber, S. Anders, Moderated estimation of fold change and dispersion for RNA-seq data with DESeq2. Genome Biol 15, 550 (2014).

36. S. Anders, W. Huber, Differential expression analysis for sequence count data. Genome Biol 11, R106 (2010).

37. Y. Benjamini, D. Drai, G. Elmer, N. Kaflcafi, I. Golani, Controlling the false discovery rate in behavior genetics research. Behav Brain Res 125, 279-284 (2001).

38. C. W. Law, Y. Chen, W. Shi, G. K. Smyth, voom: Precision weights unlock linear model analysis tools for RNA-seq read counts. Genome Biol 15, R29 (2014).

What is claimed is:

1. A method of diagnosing and treating Frontotemporal dementia (FTD) in a subject in need thereof, the method comprising:
(a) detecting a level of at least one micro RNA (miR) selected from the group consisting of hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p and hsa-miR-340-5p in a biological sample of the subject, wherein when said level of said at least one micro RNA (miR) is higher than that in a control sample, it is indicative of FTD;
(b) treating the subject with a drug which ameliorates symptoms associated with FTD.

2. A method of treating Frontotemporal dementia (FTD) in a subject in need thereof, the method comprising: treating the subject with a drug which ameliorates symptoms associated with FTD, wherein the subject has been diagnosed with FTD by detecting a level of at least one micro RNA (miR) selected from the group consisting of hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p and hsa-miR-340-5p in a biological sample of the subject, wherein when said level of said at least one micro RNA (miR) is higher than that in a control sample, it is indicative of FTD.

3. A method for selecting subjects for enrollment in a clinical trial involving treatment of Frontotemporal dementia (FTD), the method comprising:
(a) determining diagnosis of a subject by by detecting a level of at least one micro RNA (miR) selected from the group consisting of hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p and hsa-miR-340-5p in a biological sample of the subject, wherein when said level of said at least one micro RNA (miR) is higher than that in a control sample, it is indicative of FTD; and
(b) identifying the subject as being suitable for said clinical trial based on the criteria of said clinical trial.

4. The method of claim 1, wherein at least one additional micro RNA (miR) is selected from the group consisting of hsa-miR-326, hsa-miR-128-3p, hsa-miR-423-5p, hsa-miR-107, hsa-miR-23b-3p, hsa-miR-320c, hsa-miR-1306-5p, hsa-let-7d-3p, hsa-miR-378a-3p, hsa-miR-103a-3p, hsa-miR-148a-3p, hsa-miR-409-3p and hsa-miR-148b-3p.

5. The method claim 1, wherein said at least one micro RNA (miR) comprises:
(i) hsa-miR-361-5p and hsa-miR-629-5p;
(ii) hsa-miR-361-5p, hsa-miR-629-5p, hsa-miR-628-3p, hsa-miR-379-5p, hsa-miR-1-3p, hsa-miR-26a-5p, hsa-miR-125a-5p, hsa-miR-125b-5p, hsa-miR-142-5p, hsa-miR-340-5p, hsa-miR-326, hsa-miR-128-3p, hsa-miR-423-5p, hsa-miR-107, hsa-miR-23b-3p, hsa-miR-320c, hsa-miR-1306-5p, hsa-let-7d-3p, hsa-miR-378a-3p, hsa-miR-103a-3p, hsa-miR-148a-3p, hsa-miR-409-3p and hsa-miR-148b-3p;
(iii) miR-1-3p, miR-361-5p, miR-629-5p, miR-423-5p;
(iv) miR-1-3p, miR-361-5p, miR-629-5p, miR-423-5p, miR-148b-3p, miR-628-3p, miR-409-3p, miR-378a-3p, miR-125b-5p and miR-23b-3p;
(v) miR-1-3p, miR-361-5p, miR-629-5p, miR-423-5p, miR-148b-3p, miR-628-3p, miR-409-3p, miR-378a-3p, miR-125b-5p, miR-23b-3p, miR-326 and miR-103a-3p;
(vi) miR-1-3p, miR-361-5p, miR-629-5p, miR-423-5p, miR-148b-3p, miR-628-3p, miR-409-3p, miR-378a-3p, miR-125b-5p, miR-23b-3p, miR-326, miR-103a-3p, miR-142-5p, miR-125a-5p, miR-26a-5p, miR-148a-3p, miR-379-5p and miR-320c;
(vii) miR-1-3p, miR-361-5p, miR-629-5p, miR-423-5p, miR-148b-3p, miR-628-3p, miR-409-3p, miR-378a-3p, miR-125b-5p, miR-23b, miR-326, miR-103a-3p, miR-142-5p, miR-125a-5p, miR-26a-5p, miR-148a-3p, miR-379-5p, miR-320c, let-7d-3p, miR-340-5p and miR-1306-5p;
(viii) hsa-miR-361-5p, hsa-miR-629-5p, hsa-miR-26a-5p, hsa-miR-23b-3p, hsa-miR-326, hsa-miR-103a-3p, hsa-miR-379-5p, hsa-miR-320c, hsa-miR-128-3p, hsa-miR-148a-3p, hsa-miR-628-3p, hsa-miR-423-5p and hsa-let-7d-3p; or
(ix) hsa-miR-361-5p, hsa-miR-629-5p, hsa-miR-26a-5p, hsa-miR-23b-3p, hsa-miR-326, hsa-miR-103a-3p, hsa-miR-379-5p, hsa-miR-320c, hsa-miR-128-3p, hsa-miR-148a-3p, hsa-miR-628-3p, hsa-miR-423-5p and hsa-let-7d-3p.

6. The method of claim 1, further comprising retrieving said biological sample from the subject.

7. The method of claim 1, wherein said biological sample is cell-free.

8. The method of claim 1, wherein said biological sample is selected from the group consisting of a plasma, a serum and a cerebrospinal fluid sample.

9. The method of claim 1, wherein said detecting is effected by real time PCR (RT-PCR).

10. The method of claim 1, wherein said detecting is effected by next generation sequencing (NGS).

11. The method of claim 1, wherein the subject is a human being.

12. The method of claim 1, wherein said at least one micro RNA (miR) does not exceed 100 miRs.

13. The method of claim 1, wherein said at least one micro RNA (miR) does not exceed 50 miRs.

14. The method of claim 1, wherein said at least one micro RNA (miR) does not exceed 30 miRs.
